(19) United States
(12) Patent Application Publication
(10) Pub. No.: US 2020/0376003 A1
(43) Pub. Date: Dec. 3, 2020

(54) PHARMACEUTICAL COMPOSITION FOR PREVENTING OR TREATING CHOLESTEROL GALLSTONE COMPRISING URSODEOXYCHOLIC ACID (UDCA) AND OMEGA-3 FATTY ACID, AND PROCESS FOR PRODUCING THE SAME

(71) Applicant: INDUSTRY-ACADEMIC COOPERATION FOUNDATION, YONSEI UNIVERSITY, Seoul (KR)

(72) Inventors: Dong Ki LEE, Seongnam-si (KR); Sungill JANG, Seoul (KR)

(21) Appl. No.: 16/427,490

(22) Filed: May 31, 2019

(51) Int. Cl. A61K 31/575; A23L 33/105; A23L 33/12; A61K 31/202; A61K 47/44; A61P 1/16

(52) U.S. Cl. CPC A61K 31/575; A23L 33/105; A23L 33/12; A61K 31/202; A61K 47/44; A23V 2200/32; A23V 2002/00; A23V 2250/02; A23V 2250/1868; A23V 2250/187; A23V 2250/21; A61P 1/16

(57) ABSTRACT

The present invention relates to a gallstone solubilizing agent, comprising ursodeoxycholic acid (UDCA) or a pharmaceutically acceptable salt thereof; omega-3 fatty acid; and an excipient, the gallstone solubilizing agent resulting in a process which prevents cholesterol gallstones from being formed or causes cholesterol gallstones to be dissolved, a pharmaceutical composition comprising the same, and a health functional food composition comprising the same. A pharmaceutical composition prepared to have UDCA and omega-3 fatty acid at an optimal content ratio allows cholesterol to remain in a dissolved state without forming crystals, and thus results in a process which prevents cholesterol gallstones from being formed or causes cholesterol gallstones to be dissolved, thereby exhibiting increased effects of preventing or treating cholesterol gallstones.

### BACKGROUND

### 1. Field of the Invention

The present invention relates to a gallstone solubilizing agent, comprising ursodeoxycholic acid (UDCA) or a pharmaceutically acceptable salt thereof; omega-3 fatty acid; and an excipient, the gallstone solubilizing agent resulting in a process which prevents cholesterol gallstones from being formed or causes cholesterol gallstones to be dissolved, a pharmaceutical composition comprising the same, and a health functional food composition comprising the same.

### 2. Description of the Related Art

In western countries, 10% to 15% of the population has gallstones. In the United States, among benign digestive tract diseases, medical expenses due to gallstones are in the first place, and thus gallstone disease is a disease that causes huge medical expenses. In particular, cholesterol gallstone disease is so prevalent that 20 million people are receiving medical treatment due to this disease each year. As the economic level of South Korea has improved and the diet has become westernized, patients having cholesterol gallstones or mixed gallstones are increasing rapidly. According to the data released by the National Health Insurance Corporation as of February 2014, the number of patients with gallstone disease has increased by an annual average of 7.3% over the years 2007 to 2012.

Surgical treatment, such as cholecystectomy through laparotomy, is a basic therapeutic method. Medical treatment has been limited because of its low success rate; however, there are a method of dissolving gallstones, a method of crushing gallstones, and a method of using a combination of the two. The method of dissolving gallstones includes a method of using an oral gallstone dissolving agent and a method of dissolving gallstones by injecting a drug into the gallbladder. The method of crushing gallstones includes extracorporeal shock wave lithotripsy and a method in which a fistula is formed after percutaneous transhepatic gallbladder drainage and then gallstones are removed using percutaneous cholangioscopy.

With the introduction of chenodeoxycholic acid (CDCA) in the 1970s and ursodeoxycholic acid (UDCA) in the 1980s, medical treatment using an oral dissolving agent has been widely used as a replacement for high-risk laparotomic surgery. With the introduction of laparoscopic cholecystectomy in the late 1980s, due to a low success rate (30% to 40%) of gallstones dissolution and a high recurrence rate of gallstones which oral dissolving agents exhibit, laparoscopic cholecystectomy, rather than medical treatment using an oral dissolving agent, has been suggested as the main therapeutic method since its introduction, and skepticism about medical oral dissolution therapy has increased. However, although laparoscopic cholecystectomy is safer than a laparotomy method, problems related to surgery are still being raised. Laparoscopic surgery has a prevalence of 1.6% to 12% and a complication rate of 2%. The results of surgery vary greatly depending on the experience and skill of the practitioner, and even skilled practitioners experience a surgical complication rate of 0.1% to 0.5%. In addition, although cholecystectomy heals gallstones 100%, there is also a report that after surgery, about 20% of patients continue to complain of pain that was an indication for surgery. For this reason, the development of prophylactic and therapeutic methods for gallstone disease is urgently needed.

Accordingly, the present inventors have conducted experiments for effects of omega-3 fatty acid, ursodeoxycholic acid (UDCA), or omega-3 fatty acid and ursodeoxycholic acid (UDCA) in a prophylactic aspect of the gallstone disease. As a result, in a case where the omega-3 fatty acid, the ursodeoxycholic acid (UDCA), or the omega-3 fatty acid and the ursodeoxycholic acid (UDCA) are administered together with feed that forms gallstones, the group administered with omega-3 fatty acid showed lower gallstone formation than in the group administered with ursodeoxycholic acid. In addition, in a case where omega-3 fatty acid and ursodeoxycholic acid are administered in combination, inhibited gallstone formation was exhibited as compared with the group administered with omega-3 fatty acid or ursodeoxycholic acid (UDCA) alone, but there was no synergistic effect and statistically significant difference. Furthermore, gallbladder wall hypertrophy and expression degree of genes associated therewith were examined. As a result, regardless of the presence or absence of ursodeoxycholic acid, omega-3 fatty acid suppressed the gallbladder wall hypertrophy and the expression of genes associated therewith. Accordingly, the present inventors have found that omega-3 fatty acid increases the level of bile phospholipids and suppress bile mucin formation, thereby weakening gallstone formation in mice (Journal of Gastroenterology and Hepatology; 27 (2012) 1745-1751). However, this was an experiment in which omega-3 fatty acid, ursodeoxycholic acid (UDCA), or omega-3 fatty acid and ursodeoxycholic acid (UDCA) are administered together with feed that causes gallstones so as to examine prophylactic effects of the omega-3 fatty acid, the ursodeoxycholic acid (UDCA), or the omega-3 fatty acid and the ursodeoxycholic acid (UDCA). Thus, from the above description, it is not possible to know therapeutic effects of omega-3 fatty acid, ursodeoxycholic acid (UDCA), or omega-3 fatty acid and ursodeoxycholic acid (UDCA). In addition, in the previous experiments, the effects of omega-3 fatty acid were identified, and the effects of combination of omega-3 fatty acid and ursodeoxycholic acid (UDCA) were similar to those of omega-3 fatty acid. Thus, the previous experiments have a limitation that it is not possible to identify the effects of complexation of omega-3 fatty acid and ursodeoxycholic acid (UDCA).

Accordingly, the present inventors have examined therapeutic effects, on gallstone disease, of a capsule which contains omega-3 fatty acid and ursodeoxycholic acid (UDCA) with an excipient added, and therapeutic effects, on gallstone disease, of a capsule which contains, at a certain ratio, omega-3 fatty acid and ursodeoxycholic acid (UDCA) with an excipient added.

### SUMMARY OF THE INVENTION

An object of the present invention is to provide a gallstone solubilizing agent, comprising ursodeoxycholic acid (UDCA) or a pharmaceutically acceptable salt thereof; omega-3 fatty acid; and an excipient, the gallstone solubilizing agent resulting in a process which prevents cholesterol gallstones from being formed or causes cholesterol gallstones to be dissolved, a pharmaceutical composition comprising the same, and a health functional food composition comprising the same.

In order to achieve the above-mentioned object, the present invention provides a gallstone solubilizing agent, comprising ursodeoxycholic acid (UDCA) or a pharmaceutically acceptable salt thereof; omega-3 fatty acid; and an excipient.

The excipient of the present invention may be any one or more selected from the group consisting of bee's wax, wax mixture, paraffin wax, refined palm oil, hydrogenated coconut oil, hydrogenated soybean oil, hard fat, and polyethylene glycol.

In the solubilizing agent of the present invention, a weight ratio of ursodeoxycholic acid (UDCA) or a pharmaceutically acceptable salt thereof to omega-3 fatty acid is 1 (w/v %):2 (w/v %) to 1 (w/v %):8.16 (w/v %).

The gallstone of the present invention may be a cholesterol gallstone.

The composition of the present invention can induce dissolution of cholesterol gallstones, or interruption of production of cholesterol gallstone crystals.

In another embodiment of the present invention, there may be provided a pharmaceutical composition for the prevention or treatment of gallstone disease, comprising ursodeoxycholic acid (UDCA) or a pharmaceutically acceptable salt thereof; omega-3 fatty acid; and an excipient.

The excipient of the present invention may be any one or more selected from the group consisting of bee's wax, wax mixture, paraffin wax, refined palm oil, hydrogenated coconut oil, hydrogenated soybean oil, hard fat, and polyethylene glycol.

In the composition of the present invention, a weight ratio of ursodeoxycholic acid (UDCA) or a pharmaceutically acceptable salt thereof to omega-3 fatty acid may be 1 (w/v %):2 (w/v %) to 1 (w/v %):8.16 (w/v %).

The gallstone of the present invention may be a cholesterol gallstone.

The composition of the present invention can dissolve cholesterol gallstones or suppress production of cholesterol gallstone crystals.

In yet another embodiment of the present invention, there is provided a health functional food composition for dissolving gallstones or suppressing production of gallstone crystals, comprising ursodeoxycholic acid (UDCA) or a pharmaceutically acceptable salt thereof; omega-3 fatty acid; and an excipient.

The excipient of the present invention may be any one or more selected from the group consisting of bee's wax, wax mixture, paraffin wax, refined palm oil, hydrogenated coconut oil, hydrogenated soybean oil, hard fat, and polyethylene glycol.

The composition of the present invention can induce dissolution of cholesterol gallstones, or interruption of production of cholesterol gallstone crystals.

In the composition of the present invention, a weight ratio of ursodeoxycholic acid (UDCA) or a pharmaceutically acceptable salt thereof to omega-3 fatty acid may be 1 (w/v %):2 (w/v %) to 1 (w/v %):8.16 (w/v %).

The gallstone of the present invention may be a cholesterol gallstone.

A pharmaceutical composition prepared to have UDCA and omega-3 fatty acid at an optimal content ratio allows cholesterol to remain in a dissolved state without forming crystals, and thus results in a process which prevents cholesterol gallstones from being formed or causes cholesterol gallstones to be dissolved, thereby exhibiting increased effects of preventing or treating cholesterol gallstones.

### BRIEF DESCRIPTION OF THE DRAWINGS

FIG. 1 illustrates a schematic diagram for animal studies.

FIG. 2 (A) illustrates a table showing body weights and weight changes following the diet and therapeutic agent administration. (B) graphically illustrates the table of (A).

FIG. 3 illustrates anatomical changes in gallbladder and gallstones before and after the drug administration.

FIG. 4 illustrates a classification chart for gallstone size (see Takashi A et al. Cholesterol gallstone in alloxan-diabetic mice, Journal of Lipid Research, 1986).

FIG. 5 illustrates the results obtained by measuring, for each group, the gallstone size after the high-cholesterol diet and the gallstone size after the therapeutic agent administration.

FIG. 6 illustrates changes in the size of gallstones for each group.

FIG. 7 illustrates changes in the amount of gallstones before and after the therapeutic agent administration.

FIG. 8 (A) illustrates changes in the weight of gallstones between before the therapeutic agent administration (pre-treatment) and after the therapeutic agent administration (post-treatment), and (B) illustrates the results obtained by comparing the difference between before and after the treatment.

### DETAILED DESCRIPTION OF EXEMPLARY EMBODIMENTS

Hereinafter, the present invention will be described in more detail.

As described above, as dietary habits of South Koreans have been westernized and changed to a high-protein, high-fat, and high-calorie diet, a prevalence of gallstone disease, in particular, cholesterol gallstone disease, has increased. In a recent survey targeted to patients across the country, it has been shown that cholesterol gallstones account for 58.1% of all gallstones, and thus it is urgently needed to develop a therapeutic method therefor.

Accordingly, the present inventors have developed a gallstone solubilizing agent, comprising, at an optimal content ratio, ursodeoxycholic acid (UDCA) or a pharmaceutically acceptable salt thereof; omega-3 fatty acid; and an excipient. The cholesterol solubilizer prepared using the optimal content ratio allows cholesterol to remain in a dissolved state without forming crystals, and thus results in a process which prevents cholesterol gallstones from being formed or causes cholesterol gallstones to be dissolved, thereby exhibiting a synergistic effect of preventing or treating cholesterol gallstones.

The present invention provides a gallstone solubilizing agent, comprising ursodeoxycholic acid (UDCA) or a pharmaceutically acceptable salt thereof; omega-3 fatty acid; and an excipient.

Ursodeoxycholic acid (3α,7β-dihydroxy-5β-cholanoic acid, hereinafter referred to as “UDCA”) is a major component of the so-called bear gallbladder that has been used for liver and biliary diseases in South Korea, China, Japan, and the like for a long time. UDCA is one type of bile acids found in the bile, and has a molecular weight of 392.58 and a molecular formula of C₂₄H₄₀O₄. This UDCA is a drug that has been used with major clinical indications for gallstone disease and biliary diseases, chronic liver disease and liver function improvement, dyspepsia after small bowel resection, and fatty liver, from the viewpoint that UDCA has an action of cleaning the micro-biliary tract in the living body, and thus of discharging waste materials and toxic bile acids in the micro-biliary tract; an action of stabilizing the hepatic cell membrane and protecting hepatocytes; an action of increasing the hepatic blood flow; an action of suppressing absorption and biosynthesis of cholesterol; an action of dissolving gallstones and suppressing production thereof; and pharmacological activity of normalizing immune activity. In particular, in the mechanism for the effect of UDCA to dissolve cholesterol gallstones, UDCA is known to function to decrease cholesterol secretion into the bile by decreasing activity of HMG CoA reductase which is an enzyme necessary for cholesterol synthesis in the liver, to increase 7α-hydroxylase activity, and to decrease cholesterol absorption in the intestine. In addition, administration of UDCA changes cholesterol-supersaturated bile into unsaturated bile. Desaturation of bile caused by the UDCA administration improves an ability of the bile to transport cholesterol, and causes multilamellar liposomes (mesophases) having high solubility to be formed in the bile, thereby forming liquid crystals even in cholesterol-saturated bile. Thus, for the reason that liquid crystals are formed, after use of UDCA, gallstones are dissolved even in cholesterol-supersaturated bile. Furthermore, UDCA is frequently used as a therapeutic agent for cholesterol gallstones.

Omega-3 fatty acids are a family of polyunsaturated fatty acids of which a common feature is that the last double bond is located in the third C—C bond starting from the final methyl group of the fatty acid. Omega-3 fatty acids are essential fatty acids, that is, the human body cannot internally produce these fatty acids, and thus it is necessarily required to ingest such fatty acids through a food or a composition. Due to their polyunsaturated nature, omega-3 fatty acids have very special physicochemical functions (that is, very low melting point) in the human body, and therefore have been widely studied. Today, there are up to 10 omega-3 fatty acids (for example, stearic acid); and it is known that these omega-3 fatty acids are present in very small amounts in the human body, and physiological activity thereof is very low or absent, except for DHA and/or EPA. The omega-3 fatty acid used in the present invention may be omega-3-acid ethyl ester 90 containing 400 mg or more of EPA and 340 mg or more of DHA.

The excipient of the present invention may be any one or more selected from the group consisting of bee's wax, wax mixture, paraffin wax, refined palm oil, hydrogenated coconut oil, hydrogenated soybean oil, hard fat, and polyethylene glycol, with bee's wax and hydrogenated coconut oil being most preferred.

In the solubilizing agent, a weight ratio of ursodeoxycholic acid (UDCA) or a pharmaceutically acceptable salt thereof to omega-3 fatty acid is 1 (w/v %):2 (w/v %) to 1 (w/v %):8.16 (w/v %), preferably 1 (w/v %):3.06 to 1 (w/v %):6.12 (w/v %), and most preferably 1 (w/v %):4.08 (w/v %).

The gallstone may be a cholesterol gallstone.

The solubilizing agent may dissolve cholesterol gallstones or suppress production of cholesterol gallstone crystals.

The present invention may also provide a pharmaceutical composition for the prevention or treatment of gallstone disease, comprising ursodeoxycholic acid (UDCA) or a pharmaceutically acceptable salt thereof; omega-3 fatty acid; and an excipient.

The excipient of the present invention may be any one or more selected from the group consisting of bee's wax, wax mixture, paraffin wax, refined palm oil, hydrogenated coconut oil, hydrogenated soybean oil, hard fat, and polyethylene glycol.

The pharmaceutical composition may further comprise a pharmaceutically acceptable carrier. As used herein, the term “pharmaceutically acceptable carrier” refers to a carrier or diluent that does not irritate an organism and does not inhibit the biological activity and properties of the administered compound. For the composition to be prepared into a liquid solution, as the pharmaceutically acceptable carrier, saline, sterile water, Ringer's solution, buffered saline, albumin injection solution, dextrose solution, maltodextrin solution, glycerol, and ethanol, which are sterile and biocompatible, and an admixture of one or more thereof may be used. If necessary, other conventional additives such as an antioxidant, a buffer, and a bacteriostatic agent may be added to the composition. In addition, a diluent, a dispersant, a surfactant, a binder, and a lubricant may be additionally added to the composition so that the composition is prepared into injectable solutions, such as aqueous solutions, suspensions, and emulsions, pills, capsules, granules, or tablets.

The pharmaceutical composition may have any one formulation selected from the group consisting of tablets, pills, powders, granules, capsules, suspensions, solutions, emulsions, syrups, sterilized aqueous solutions, non-aqueous solutions, suspensions, lyophilized preparations, and suppositories. Most preferably, the formulation may be capsules.

An appropriate dosage of the pharmaceutical composition of the present invention varies depending on factors such as the preparation method, the administration method, the patient's age, body weight, sex, pathological condition, diet, administration time, administration route, excretion rate, and reaction sensitivity. An ordinarily skilled practitioner can easily determine and prescribe the dosage that is effective for the desired treatment or prevention. According to a preferred embodiment of the present invention, the daily dosage of the pharmaceutical composition of the present invention is 0.001 to 100 mg/kg.

In the composition of the present invention, a weight ratio of ursodeoxycholic acid (UDCA) or a pharmaceutically acceptable salt thereof to omega-3 fatty acid is 1 (w/v %):2 (w/v %) to 1 (w/v %):8.16 (w/v %), preferably 1 (w/v %):3.06 (w/v %) to 1 (w/v %):6.12 (w/v %), and most preferably 1 (w/v %):4.08 (w/v %).

The gallstone of the present invention may be a cholesterol gallstone.

The composition of the present invention can induce dissolution of cholesterol gallstones, or interruption of production of cholesterol gallstone crystals. Administration of omega-3 fatty acid increases phospholipids in the bile, and administration of UDCA increases bile salts in the bile to balance with the increased cholesterol in the bile, so that cholesterol does not form crystals and remains in a dissolved state, thereby resulting in a process which prevents cholesterol gallstones from being formed or causes cholesterol gallstones to be dissolved.

In still yet another embodiment of the present invention, there may be provided a health functional food composition for dissolving gallstone or suppressing production of gallstone crystals, comprising ursodeoxycholic acid (UDCA) or a pharmaceutically acceptable salt thereof; omega-3 fatty acid; and an excipient.

The excipient of the present invention may be any one or more selected from the group consisting of bee's wax, wax mixture, paraffin wax, refined palm oil, hydrogenated coconut oil, hydrogenated soybean oil, hard fat, and polyethylene glycol.

In the composition, a weight ratio of ursodeoxycholic acid (UDCA) or a pharmaceutically acceptable salt thereof to omega-3 fatty acid may be 1 (w/v %):2 (w/v %) to 1 (w/v %):8.16 (w/v %), preferably 1 (w/v %):3.06 to 1 (w/v %):6.12 (w/v %), and most preferably 1 (w/v %):4.08 (w/v %).

The gallstone may be a cholesterol gallstone.

### EXAMPLES

### Example 1. Creation of Mouse Model for Identification of Treatment on Cholesterol Gallstone Formation

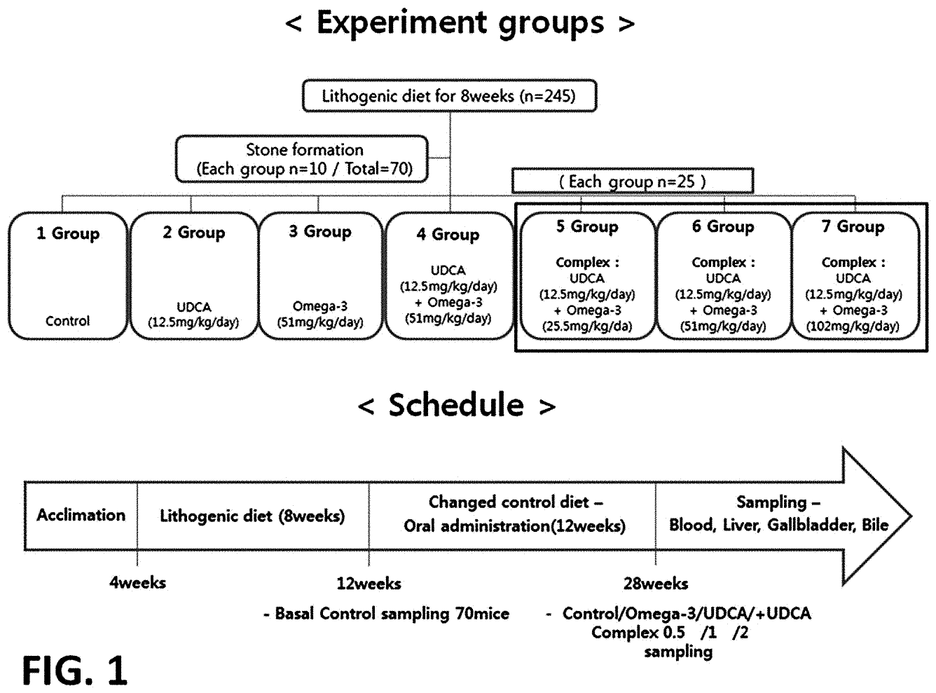

An overall schematic diagram of the animal experiment is as illustrated in FIG. 1. This will be described below in more detail. C57BL/6J male mice were purchased from Central Lab. Animal Inc. The mice were allowed to have a 4-week compliance period under a condition of controlled light (a cycle of 12-hour light and 12-hour darkness in which darkness is from 6:30 pm to 6:30 am), temperature (22±4° C.), and humidity (50%±20%). The mice had ad libitum access to water (autoclaved distilled water) and were fed commercial feed (Scientific Animal Food & Engineering/+40RMM) containing 2.9% fat, 15.2% protein, and 60.7% carbohydrate. In addition, a lithogenic diet (DYET #102136; Dyets, Inc., Bethlehem, Pa., USA) containing cholesterol (10 g/kg) and cholic acid (5 g/kg) was given for 8 weeks, so that mice with gallstones formed due to a high-cholesterol diet were created.

The mice with gallstones formed due to a high-cholesterol diet were divided into the following groups and experiments were conducted for 12 weeks:

\textcircled{1} Normal diet

\textcircled{2} Normal diet+UDCA (12.5 mg/kg/day) administration

\textcircled{3} Normal diet+omega-3 fatty acid (51 mg/kg/day) administration

\textcircled{4} Normal diet+UDCA (12.5 mg/kg/day)+omega-3 fatty acid (51 mg/kg/day) administration

\textcircled{5} Normal diet+Complex 0.5 (12.5 mg/kg/day of UDCA+25.5 mg/kg/day of omega-3 fatty acid)

\textcircled{6} Normal diet+Complex 1.0 (12.5 mg/kg/day of UDCA+51 mg/kg/day of omega-3 fatty acid)

\textcircled{7} Normal diet+Complex 2.0 (12.5 mg/kg/day of UDCA+102 mg/kg/day of omega-3 fatty acid)

Each of the mice of the control group \textcircled{1} was administered, using a sonde, 0.3 ml of a mixture of 0.75% Tween 80 and distilled water. The mice of the groups \textcircled{2} to \textcircled{4} were administered, using a sonde, a product obtained by diluting 12.5 mg/kg/day of UDCA and 51 mg/kg/day of omega-3 fatty acid in 0.75% Tween 80. For the mice of the groups \textcircled{5} to \textcircled{7} to be administered the complexes, the respective dosages (12.5 mg/kg/day of UDCA, 25.5 mg/kg/day, 51 mg/kg/day, and 102 mg/kg/day of omega-3 fatty acid) and an excipient were prepared in compliance with a soft capsule manufacturing process. According to the soft capsule manufacturing process, UDCA was weighed to be contained in an amount of 7% to 26% by weight based on the total weight, and the excipient (hydrogenated coconut oil, bee's wax, lecithin) and omega-3 fatty acid were mixed so that the excipient was contained in an amount of 21% to 29.54% by weight and omega-3 fatty acid was contained in an amount of 52% to 63% by weight, based on the total weight. The mixture was dissolved and cooled, and the resultant was mixed with the UDCA to produce a mixture. Then, the mixture was subjected to milling (homogenization, particle standardization) using the Commitrol mill, sieving, and defoaming (removal of foam), to prepare a suspension so as to be suitable for forming a soft capsule. Then, gelatin and an accelerator, a flavoring agent, a coloring agent, and the like were mixed, swollen, dissolved, and defoamed, to prepare a gelatin capsule base. The gelatin capsule base was filled with the suspension through a forming machine, to prepare capsules. The capsules were diluted in 0.75% Tween 80 and administered using a sonde.

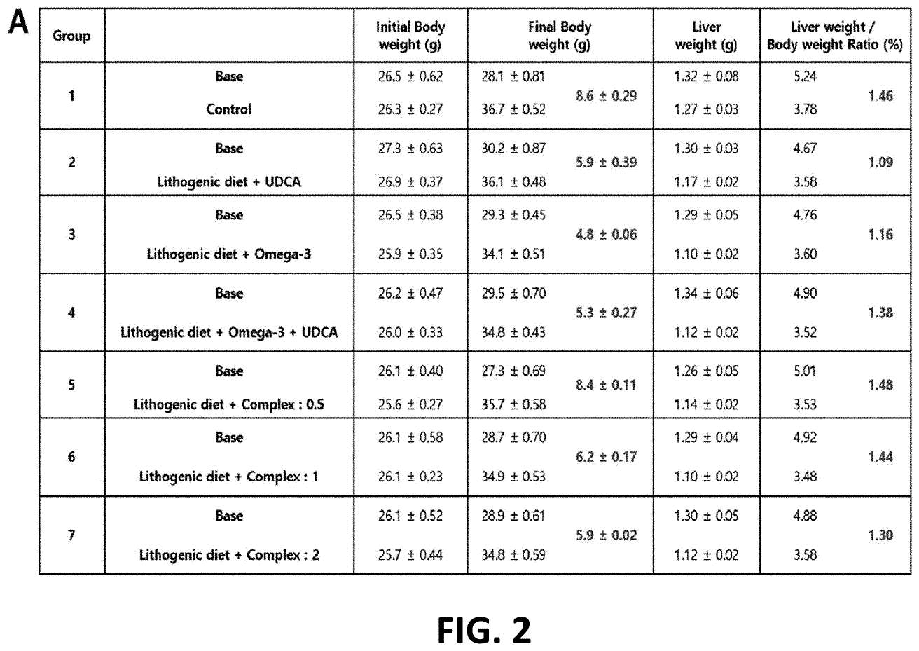

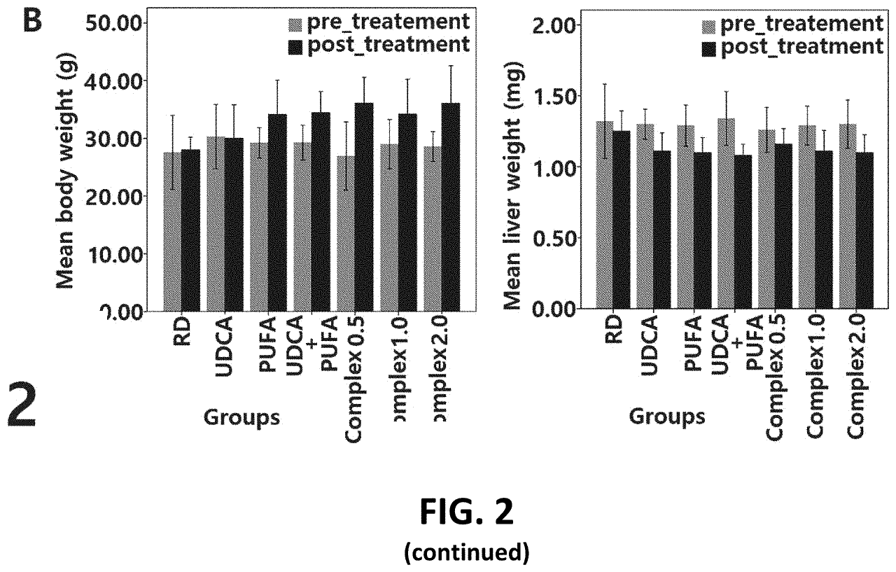

After 12 weeks of drug administration, animals were sacrificed and gallbladder tissue was obtained. Experiments were conducted using the gallbladder tissue. After the high-cholesterol diet, body weight and liver weight were measured; and after the normal diet and therapeutic agent administration, body weight and liver weight were measured (FIG. 2). There was no statistical significance, among the respective groups, in terms of changes in the body weight or liver weight over time following the diet or therapeutic agent administration, and there was no change in the liver weight relative to the body weight. From the present results, it was found that the therapeutic agent does not cause systemic complications or life-threatening problems in the experimental groups.

### Example 2. Changes in Gallbladder and Gallstones Before and after Drug Administration

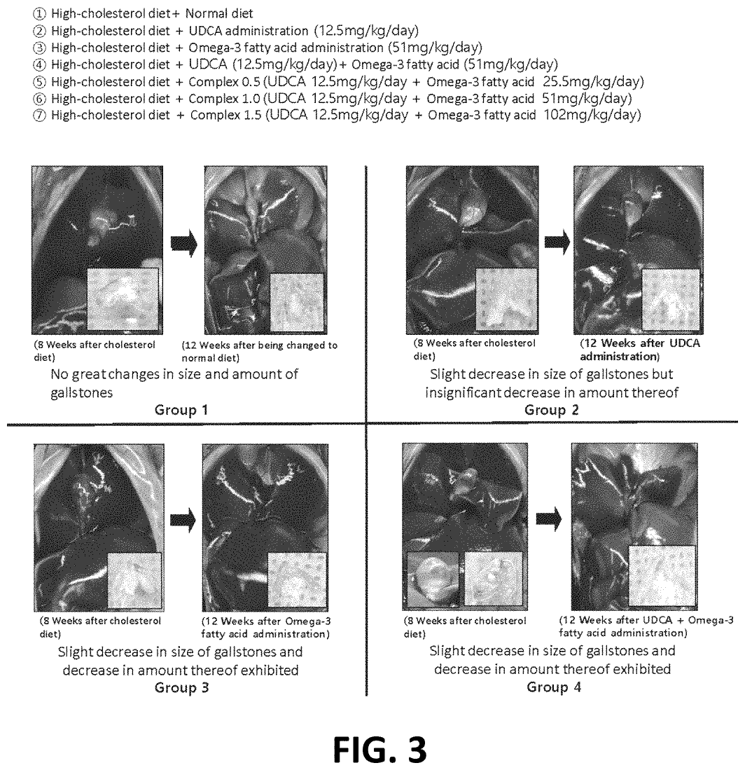

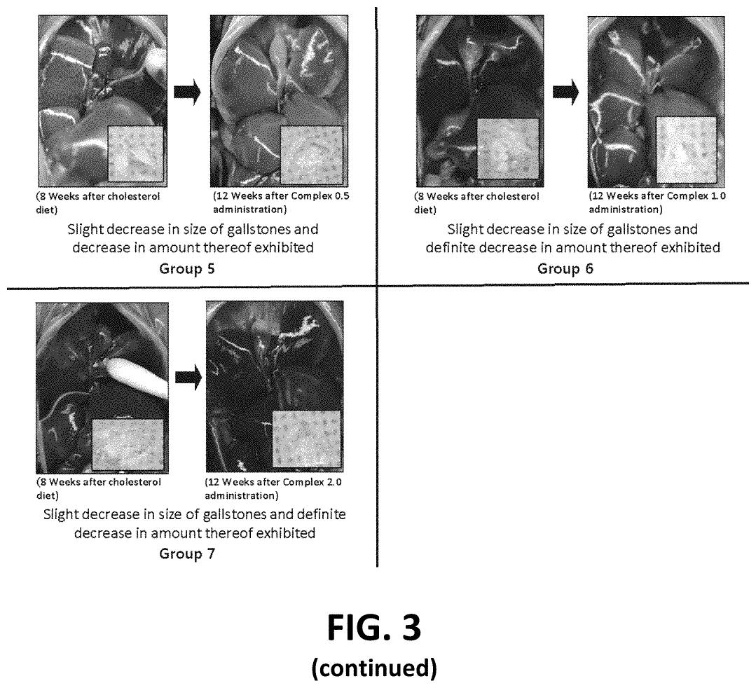

As illustrated in FIG. 3, in the mice of the group \textcircled{1}, there were no great changes in the size and amount of gallstones after being converted to the normal diet following the high-cholesterol diet. In the mice of the group \textcircled{2}, after the UDCA administration, the size of gallstones was slightly decreased and the amount thereof was insignificantly decreased. In the mice of the group \textcircled{3}, after the omega-3 fatty acid administration, the size of gallstones was slightly decreased and a decrease in the amount thereof was also exhibited. In the mice of the group \textcircled{4}, after the UDCA (12.5 mg/kg/day)+omega-3 fatty acid (51 mg/kg/day) administration, the size of gallstones was slightly decreased and a decrease in the amount thereof was also exhibited. In the mice of the group \textcircled{5}, after the Complex 0.5 (12.5 mg/kg/day of UDCA+25.5 mg/kg/day of omega-3 fatty acid) administration, the size of gallstones was slightly decreased and a decrease in the amount thereof was also exhibited. In the mice of the group \textcircled{6}, after the Complex 1.0 (12.5 mg/kg/day of UDCA+51 mg/kg/day of omega-3 fatty acid) administration, the size of gallstones was slightly decreased and a definite decrease in the amount thereof was also exhibited. In the mice of the group \textcircled{7}, after the Complex 2.0 (12.5 mg/kg/day of UDCA+102 mg/kg/day of omega-3 fatty acid) administration, the size of gallstones was slightly decreased and a decrease in the amount thereof was also exhibited; however, despite omega-3 fatty acid was administered twice as compared with the group \textcircled{6}, the size was not decreased in a concentration-dependent manner in terms of the effect.

In other words, insignificant changes in the size of gallstones were exhibited when UDCA or omega-3 fatty acid, or an admixture of the two drugs was used. However, in the groups (groups \textcircled{5}, \textcircled{6}, and \textcircled{7}) using the complex which is a composite of the two drugs, there was a significant decrease in the size of gallstones. In addition, a decrease in the size and amount of gallstones was not exhibited in a concentration-dependent manner for omega-3 fatty acid. Unusually, in the mice of the group \textcircled{6}, a definite decrease in the amount was also exhibited. As a result, it was found that the effect is not exhibited in a concentration-dependent manner for omega-3 fatty acid, and is rather increased in a range of specific content ratios of UDCA to omega-3 fatty acid.

### Example 3. Size Analysis of Gallstones Before and after Treatment

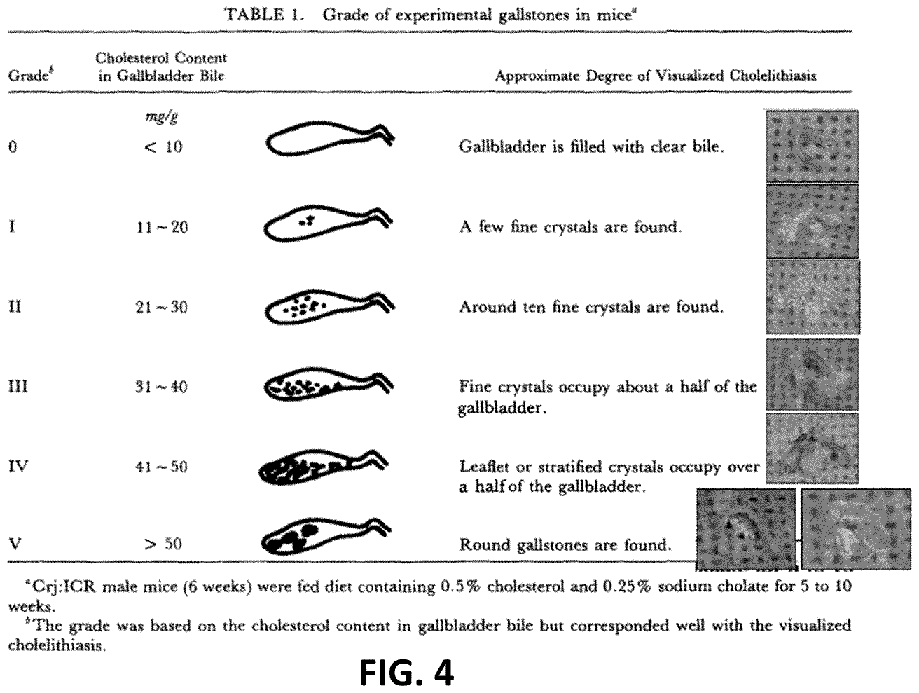

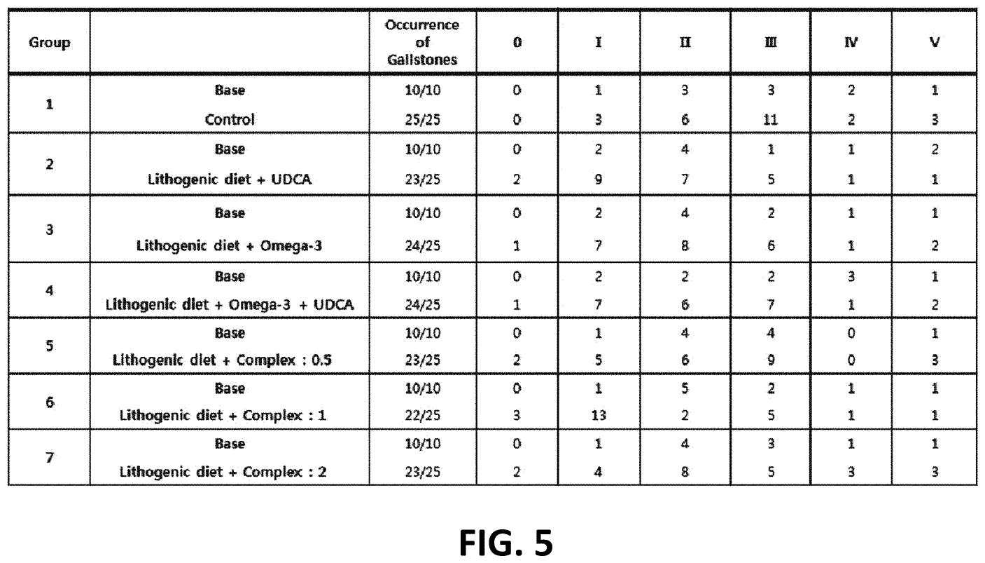

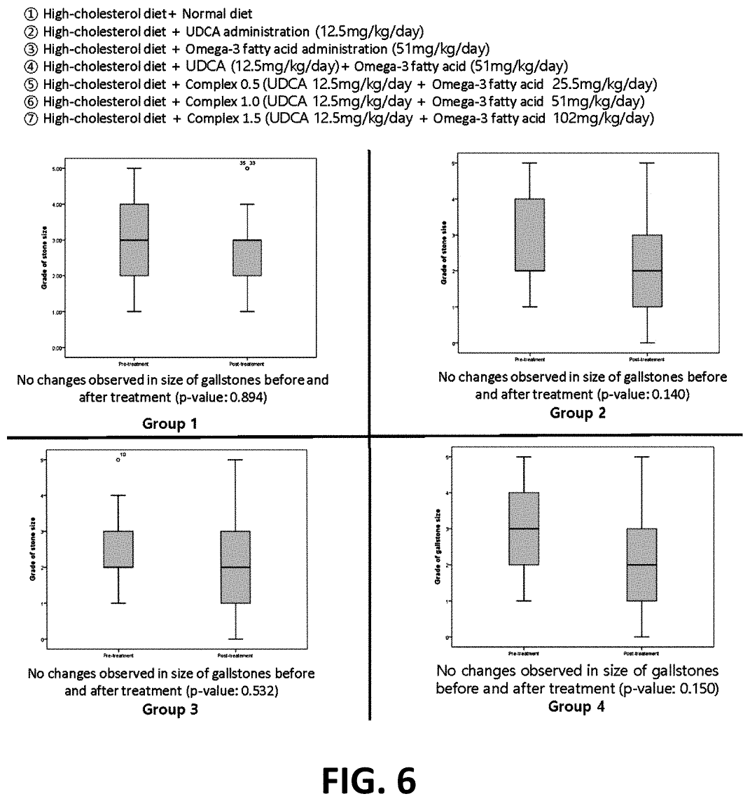

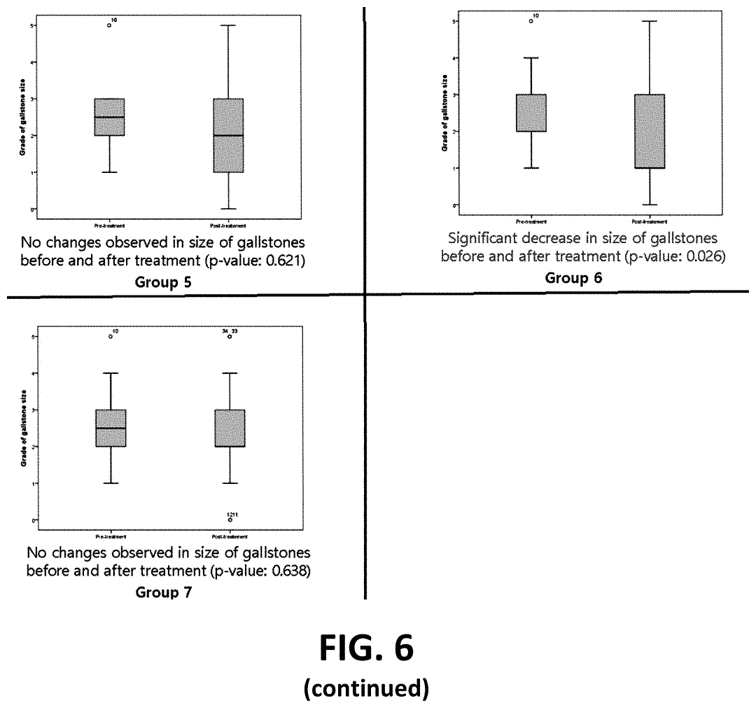

The size of gall stones in the gallbladder tissue obtained in Example 1 was analyzed on the basis of FIG. 4 (FIGS. 5 and 6). There was no change in size when UDCA or omega-3 fatty acid, or an admixture of the two drugs was used. However, in the groups (groups \textcircled{5}, \textcircled{6}, and \textcircled{7}) using the complex which is a composite of the two drugs, there was a significant decrease in the size of gallstones. The size of gallstones was significantly decreased in the group \textcircled{6} using the Complex 1.0, and this effect was not observed when other drugs were administered. As a result, it was found that the effect is not exhibited in a concentration-dependent manner, and is rather increased in a range of specific content ratios of UDCA to omega-3 fatty acid.

### Example 4. Changes in Gallstone Weight Before and after Treatment

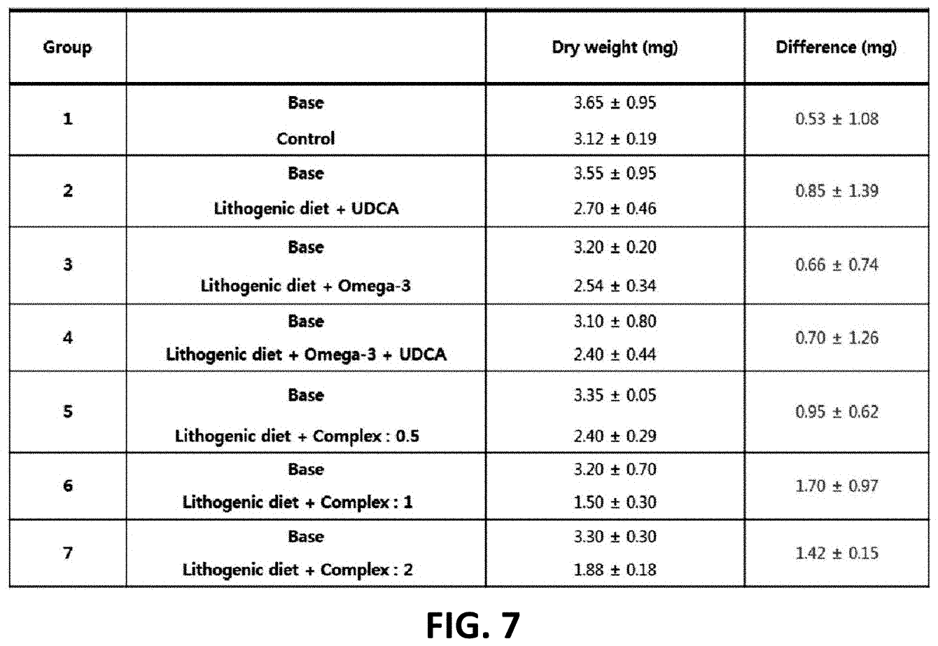

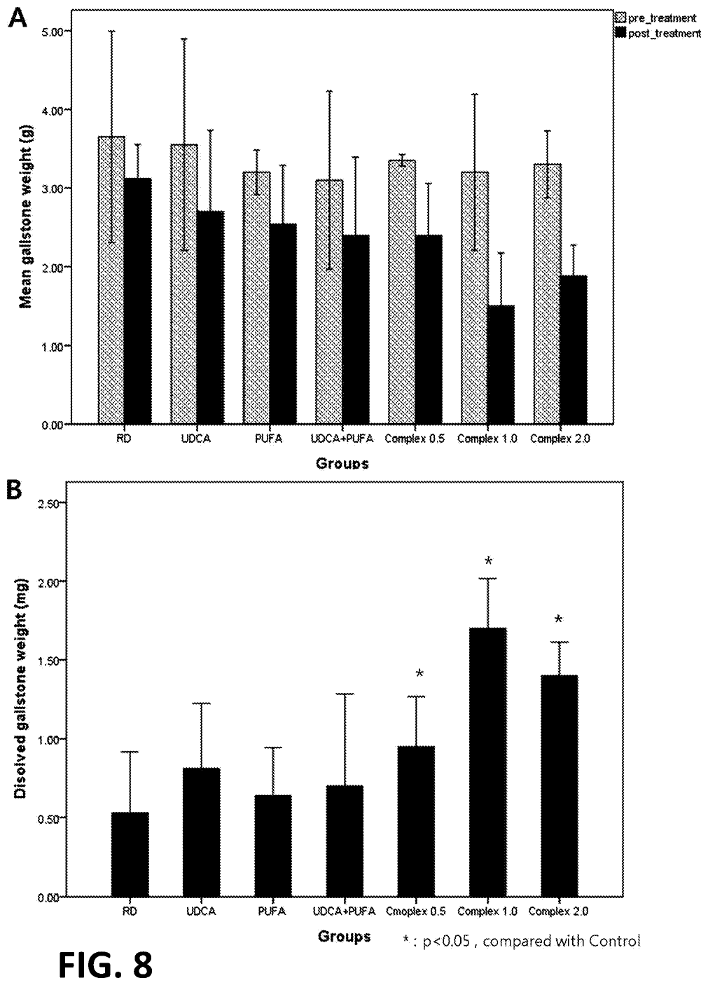

Changes in the weight of liver gallstones after the high-cholesterol diet (base) and after the therapeutic agent administration were analyzed (FIGS. 7 and 8). As can be seen from FIG. 7, the results obtained by measuring changes in the weight of liver gallstones before the therapeutic agent administration (pre-treatment) and after the therapeutic agent administration (post-treatment) showed that the amount of gallstones was decreased in all groups after the treatment, and the highest decrease was exhibited in the group \textcircled{6} using the Complex 1.0. Surprisingly, even when the amount of the complex was increased twice, changes in the weight of gallstones were not increased in a concentration-dependent manner, and was rather decreased. From this, it was found that a simple increase of the amount of the complex does not cause changes in the weight of gallstones, and UDCA and omega-3 fatty acid should be administered at a certain content ratio to have a synergistic effect for decrease in the weight of gallstones.

What is claimed is:

1. A gallstone solubilizing agent, comprising:
ursodeoxycholic acid (UDCA) or a pharmaceutically acceptable salt thereof;
omega-3 fatty acid; and
an excipient.

2. The gallstone solubilizing agent according to claim 1, wherein the excipient is any one or more selected from the group consisting of bee's wax, wax mixture, paraffin wax, refined palm oil, hydrogenated coconut oil, hydrogenated soybean oil, hard fat, and polyethylene glycol.

3. The gallstone solubilizing agent according to claim 1, wherein in the gallstone solubilizing agent, a weight ratio of the ursodeoxycholic acid (UDCA) or a pharmaceutically acceptable salt thereof to the omega-3 fatty acid is 1 (w/v %):2 (w/v %) to 1 (w/v %):8.16 (w/v %).

4. The gallstone solubilizing agent according to claim 1, wherein the gallstone is a cholesterol gallstone.

5. The gallstone solubilizing agent according to claim 1, wherein the solubilizer dissolves cholesterol gallstones or suppresses production of cholesterol gallstone crystals.

6. A pharmaceutical composition for the prevention or treatment of gallstone disease, comprising:
ursodeoxycholic acid (UDCA) or a pharmaceutically acceptable salt thereof;
omega-3 fatty acid; and
an excipient.

7. The pharmaceutical composition according to claim 6, wherein the excipient is any one or more selected from the group consisting of bee's wax, wax mixture, paraffin wax, refined palm oil, hydrogenated coconut oil, hydrogenated soybean oil, hard fat, and polyethylene glycol.

8. The pharmaceutical composition according to claim 6, wherein in the composition, a weight ratio of the ursodeoxycholic acid (UDCA) or a pharmaceutically acceptable salt thereof to the omega-3 fatty acid is 1 (w/v %):2 (w/v %) to 1 (w/v %):8.16 (w/v %).

9. The pharmaceutical composition according to claim 6, wherein the gallstone is a cholesterol gallstone.

10. The pharmaceutical composition according to claim 6, wherein the composition dissolves cholesterol gallstones or suppresses production of cholesterol gallstone crystals.

11. A health functional food composition for dissolving gallstones or suppressing production of gallstone crystals, comprising:
ursodeoxycholic acid (UDCA) or a pharmaceutically acceptable salt thereof;
omega-3 fatty acid; and
an excipient.

12. The health functional food composition according to claim 11, wherein the excipient is any one or more selected from the group consisting of bee's wax, wax mixture, paraffin wax, refined palm oil, hydrogenated coconut oil, hydrogenated soybean oil, hard fat, and polyethylene glycol.

13. The health functional food composition according to claim 11, wherein in the composition, a weight ratio of the ursodeoxycholic acid (UDCA) or a pharmaceutically acceptable salt thereof to the omega-3 fatty acid is 1 (w/v %):2 (w/v %) to 1 (w/v %):8.16 (w/v %).

14. The health functional food composition according to claim 11, wherein the gallstone is a cholesterol gallstone.
